(19) United States
(12) Plant Patent Application Publication
(10) Pub. No.: US 2013/0125271 P1
(43) Pub. Date: May 16, 2013

(54) Pear tree name 'PREMP33'

(50) ;

(71) Applicants: Roy Hart, Motueka (NZ); Lester Raymond Brewer, Motueka (NL)

(72) Inventors: Roy Hart, Motueka (NZ); Lester Raymond Brewer, Motueka (NL)

(21) Appl. No.: 13/136,644

(22) Filed: Aug. 4, 2011

Related U.S. Application Data
(60) Provisional application No. 61/401,029, filed on Aug. 6, 2010.

(51) Int. Cl. A01H 5/00

(52) U.S. Cl. PLT/176

(57) ABSTRACT

A new and distinct pear tree named ‘PremP33’ is disclosed. The new pear is notable for its large fruit size, long shelf life, and texture.

### CROSS-REFERENCE TO RELATED APPLICATIONS

This is a Non-Provisional Plant Patent Application based upon Provisional Patent Application No. 61/401,029 and claims priority under 35 U.S.C. 199(e).

### STATEMENT REGARDING FEDERALLY SPONSORED RESEARCH OR DEVELOPMENT

None.

### LATIN NAME OF THE GENUS AND SPECIES OF THE PLANT CLAIMED

Pyrus communis

### VARIETY DENOMINATION

‘PREMP33’

### BRIEF DESCRIPTION OF THE PHOTOGRAPHS

FIG. 1 shows a ‘PremP33’ pear tree;

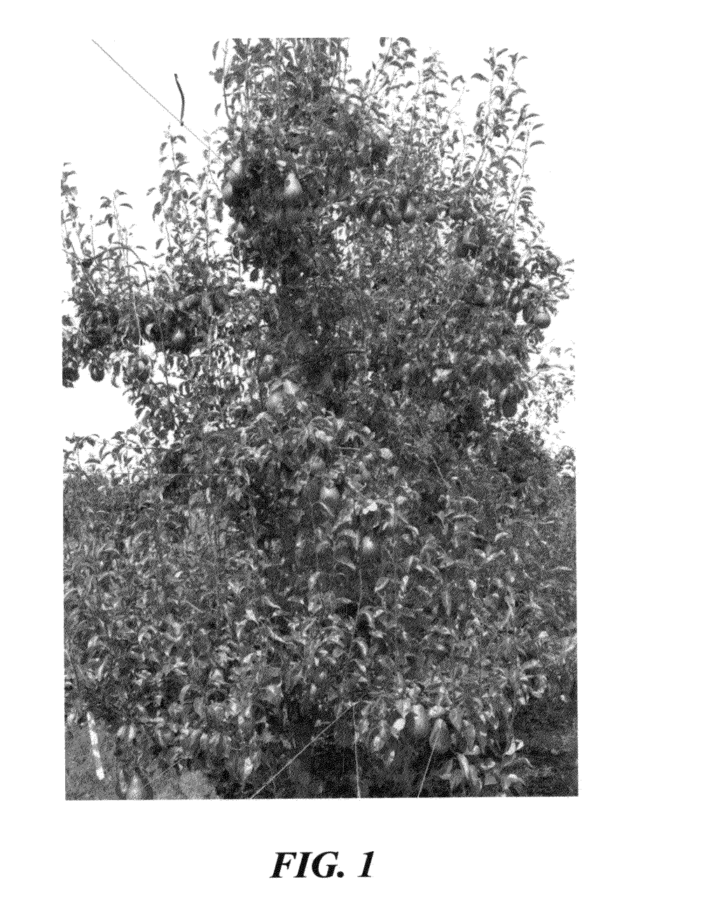

FIG. 2 shows the fruit of a ‘PremP33’ pear tree;

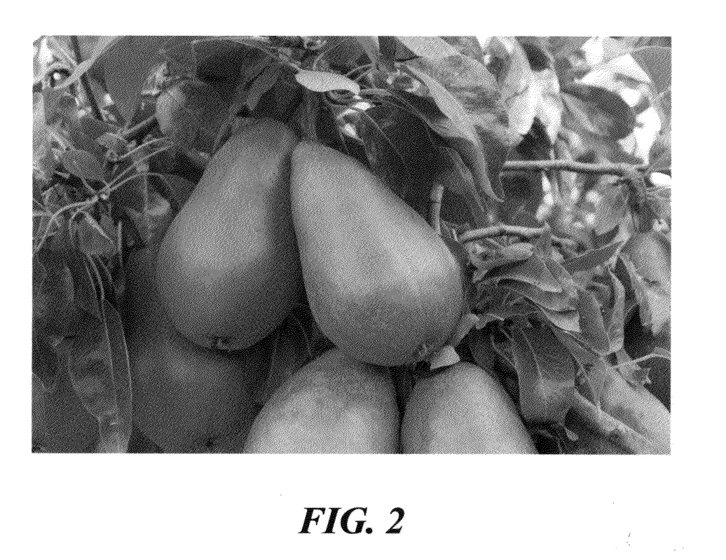

### BRIEF DESCRIPTION OF THE VARIETY

The new variety of pear tree ‘PremP33’ was created in the course of a planned breeding program carried out at The New Zealand Institute for Plant and Food Research, Hawkes Bay, New Zealand. ‘PremP33’ originated as a result of a controlled cross of ‘Patrick Barry’ (not patented) and ‘Conference’ (not patented). ‘PremP33’ was selected as a single plant from among a population of seedlings derived from the parents, and was selected for its large fruit size, long shelf life, buttery texture, fruit shape and early flowering. Asexual propagation by budding was first carried out in Nelson, New Zealand. The variety has since been observed over a number of asexually propagated generations, and has been found to remain true to type.

### DETAILED BOTANICAL DESCRIPTION OF THE VARIETY

The following detailed botanical description is based on observations made during the 2010 growing season at Parker, Wash. All colors are described according to The Royal Horticultural Society Colour Chart. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average.
Tree:
   Vigor.—Good.
   Habit.—Upright-spreading.
   Size.—Height 283 cm, spread 122 cm.
   Trunk.—Diameter at 30 cm above graft union 6 cm.
   Bark texture.—smooth.
   Bark color.—under color 166B, over color N200D.
   Lenticels.—diameter — 0.1 cm.
   Lenticel color.—164A.
Branch:
   Size.—Length 41 cm, diameter 0.8 cm at 1 m above graft union Crotch angle.
   Relative to trunk.—50°.
   Branch color.—166B.
   Lenticel diameter.—0.1 cm.
   Lenticel color.—164A.
   Lenticel density.—18 per square inch.
Dormant one-year-old shoot:
   Pubescence.—smooth.
   Size.—diameter 0.7 cm, length 59 cm.
   Internode length.—3 cm.
   Color.—166B; Internode length — 3 cm.
   Lenticels.—quantity 34 per square inch, color 196C, diameter 0.1 cm.
Flower buds:
   Quantity per spur.—2 to 3 clusters per branch.
   Shape.—Conical.
   Size.—Length 0.6 cm; Diameter 0.8 cm.
   Color.—144C.
Flowers:
   Color at balloon stage.—N155A with 65A pink in petal.
   Diameter of open flower.—2 cm.
   Relative position of petal margin.—Cupped blossoms, petals not touching.
   Depth.—1.4 cm.
   Number per cluster.—8-11 blossoms in a cluster.
Petals:
   Number per flower.—5.
   Shape.—Oval; Apex rounded; Base slightly pointed.
   Size.—Length 1.6 cm; Diameter 0.1 cm at base, 0.4 cm at apex, 0.9 cm at widest point.
   Margin.—Ruffled.
   Color.—Upper surface N155A; Lower surface N155A.
Pistils:
   Size.—0.8 cm.
   Color.—145D.
Anthers:
   Quantity.—11.
   Size.—0.1 cm.
   Pollen.—produced.
   Color.—N200A.
Stigma:
   Size.—Diameter 0.1 cm.
   Color.—153D.
Style:
   Size.—Diameter 0.1 cm.
   Color.—155D.
Ovary:
   Size.—Diameter 0.3 cm.
   Color.—145A.
Bloom:
   Date of first bloom.—April 17^{th} in Parker, Wash.
   Date of full bloom.—April 22^{nd} in Parker, Wash.
Pedicel:
   Size.—Length 2.4 cm; Diameter 0.2 cm.
   Color.—145A.
Sepals:
   Quantity.—5.
   Size.—Length 0.7 cm; Diameter 0.2 cm.
   Color.—145B.
Leaf:
   Attitude in relation to shoot.—Upward.
   Blade margin.—Serrated.
   Size.—Length 7.2 cm, Width 3.9 cm.
   Color.—Upper surface 144A; Lower surface 144D.
Petiole:
   Size.—Length 1.5 cm; Diameter 0.1 cm.
   Color.—145B.
   Attitude in relation to shoot.—80°.
Petiole:
   Length.—1.5 cm.
   Diameter.—0.1 cm.
   Color.—145B.
Fruit:
   Quantity per cluster.—2-3.
   Size.—Length 116 mm; Diameter at widest point 65 mm.
   Aperture of eye.—18.8 mm.
   Depth of eye.—7.3 mm.
   Sepal size.—Length 5.3 mm; Width 3.9 mm.
   Sepal color.—144A.
   Bloom of skin.—None.
   Greasiness of skin.—None.
   Color of skin.—Solid color 144A with 35% spots of russet color 199A.
   Flesh texture.—Fine, melting like an avocado.
   Juiciness.—Juicy, sweet.
   Flesh color.—155C.
   Stem color.—N199B.
   Seeds.—None.
Harvest:
   Harvest date range.—September 25^{th} through October 15^{th} in Parker, Wash.

We claim:

1. A new and distinct pear tree substantially as shown and described herein.
